(19) United States
(12) Patent Application Publication
(10) Pub. No.: US 2010/0105570 A1
(43) Pub. Date: Apr. 29, 2010

(54) Multi-Chamber Pretreatment Reactor for High Throughput Screening of Biomass

(75) Inventors: Michael H. Studer, Riverside, CA (US); Charles E. Wyman, Riverside, CA (US); Melvin P. Tucker, Golden, CO (US); Michael J. Selig, Golden, CO (US); Roman Brunecky, Golden, CO (US); Michael E. Himmel, Golden, CO (US); Stephen R. Decker, Golden, CO (US)

Correspondence Address: PAUL J WHITE, PATENT COUNSEL;NATIONAL RENEWABLE ENERGY LABORATORY (NREL), 1617 COLE BOULEVARD, MS 1734, GOLDEN, CO, 80401-3393, US

(73) Assignees: ALLIANCE FOR SUSTAINABLE ENERGY, LLC, Golden, CO (US); THE REGENTS OF THE UNIVERSITY OF CALIFORNIA, Oakland, CA (US)

(21) Appl. No.: 12/576,993

(22) Filed: Oct. 9, 2009

Related U.S. Application Data
(60) Provisional application No. 61/104,237, filed on Oct. 9, 2008.
(60) Provisional application No. 61/118,884, filed on Dec. 1, 2008.

(51) Int. Cl. C40B 30/04; C40B 60/12; C12P 19/04

(52) U.S. Cl. 506/9; 506/39; 435/101

(57) ABSTRACT

The disclosure provides reactors for rapid pretreatment of multiple biomass samples in a simple, process-driven, high throughput screening assay. This disclosure also provides methods and systems for rapid, high-throughput pretreatment and subsequent enzyme hydrolysis testing of multiple biomass samples.

### CROSS-REFERENCE TO RELATED APPLICATIONS

This application claims priority to U.S. Provisional Application Nos. 61/104,237, filed Oct. 9, 2008, and 61/118,884, filed Dec. 1, 2008, the contents of which are incorporated by reference in their entirety.

### CONTRACTUAL ORIGIN

The United States Government has rights in this invention under Contract No. DE-AC36-08G028308 between the United States Department of Energy and the Alliance for Sustainable Energy, LLC, manager and operator of the National Renewable Energy Laboratory.

### BACKGROUND

Biological conversion of lignocellulosic materials provides a sustainable and renewable route for the production of electric power and liquid transportation fuels. Current technology for biomass conversion to biofuels, e.g., bioethanol, involves the integration of three major steps: particle size reduction and pretreatment, enzymatic hydrolysis, and fermentation of the lignocellulosic sugars.

Heat and/or chemical pretreatment of biomass are generally considered to be prerequisite steps in the conversion of biomass to free sugars. The pretreatment step alters the biomass in various ways depending on the method used, but the result is that biomass subjected to pretreatment is more amenable to enzymatic digestion than the raw or non-pretreated starting material. The effectiveness of biomass pretreatment is dependent upon several factors, including time, temperature, pressure, and the strength and composition of the chemical catalyst employed, but is also affected by the composition of the biomass itself.

Biomass composition varies significantly by species, but genetic variants of a single species can also present different levels of susceptibility to pretreatment. In addition, environmental factors during growth can influence the composition and structure of plant biomass, affecting pretreatment and enzymatic digestibility. These factors can include, but are not limited to, location, soil type, water availability, light levels, and nutrient variability. These variables generate a huge number of permutations in the level of susceptibility of biomass to pretreatment and enzyme digestibility.

A typical biomass pretreatment method requires a series of manual steps including weighing and loading the biomass; measuring and adding the liquid catalyst; assembling and sealing the reaction chamber; heating and cooling the chamber; unloading the reactor contents; separating the liquid and solid fractions; neutralizing, washing, or conditioning the samples; and cleaning the reactor. In order to automate this process, a reactor designed to work with standard automation robots is needed. However, many of the above steps are not amenable to automation with off-the-shelf products and standard protocols. The present exemplary reactors address many of the shortcomings found in prior biomass pretreatment processes.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Embodiments herein provide multi-chamber reactors for high throughput screening of biomass samples comprising a plurality of wells for holding biomass samples and a plurality of ports located around the sample wells that allow for rapid and even heating and cooling of the sample wells.

In certain embodiments, the plurality of wells for holding biomass samples comprises a plate comprising 96 wells and a plurality of ports disposed around the 96 wells.

In other embodiments, the multi-chamber reactor comprises a top plate and a bottom plate, and the plurality of wells for holding biomass samples are composed of a non-corrosive heat stable material and are disposed between the top plate and bottom plate. In some embodiments, the plurality of wells for holding biomass samples comprise cups composed of a non-corrosive heat stable material affixed to the bottom plate. In further embodiments, the multi-chamber reactor further comprises a means for compressing the top plate to the plurality of wells to form a seal.

In various embodiments, the reactor is constructed of a material that can withstand temperatures of at least 120° C. and pH values of about 1 to about 13, or is constructed of a metal, ceramic material, or carbon-based nanomaterial or aluminum, nickel, titanium, stainless steel, or any alloy thereof. In some embodiments, the reactor further comprises a coating of gold, nickel or titanium oxide.

In certain embodiments, the multi-chamber reactor further comprises an external clamping system or at least one gasket.

The present disclosure also provides methods for high throughput screening of biomass samples comprising placing at least one biomass sample into one or more wells of at least one multi-chamber reactor, adding water or catalyst solution to the wells of the reactor, and heating the reactor. These methods may further comprise adding at least one enzyme to the reactor wells to hydrolyze the biomass sample.

The present disclosure further provides methods for treating biomass samples comprising providing a slurry of cellulosic biomass to one or more wells of at least one multi-chamber reactor, and incubating the reactor at a temperature sufficient to open the biomass structure and release or break down hemicelluloses.

In some embodiments, the slurry is a dilute slurry of about 1 to 2% w/w. In further embodiments, the reactor is incubated at a temperature between about 120 and 200 degrees C., and this incubation may be accomplished by contacting the reactor with hot air, steam, hot sand, convection heat, or hot oil. These methods may further comprise adjusting the pH of the slurry prior to incubation or removing the biomass after incubation and extracting the sugar.

Embodiments also provide systems for high throughput screening of biomass samples comprising at least one multi-chamber reactor and a steam chamber.

In addition to the exemplary aspects and embodiments described above, further aspects and embodiments will become apparent by reference to the drawings and by study of the following descriptions.

### BRIEF DESCRIPTION OF THE DRAWINGS

Exemplary embodiments are illustrated in the drawings. The embodiments and figures disclosed herein are to be considered illustrative rather than limiting.

FIG. 1 is a schematic representing co-hydrolysis and the traditional pretreatment and enzymatic hydrolysis approaches. In the traditional approach to characterize pretreatment and enzymatic hydrolysis, the solids and the liquid are separated after pretreatment, which is not the case for the new co-hydrolysis approach

FIG. 2 shows a comparison of the conventionally filtered, washed and hydrolyzed pretreated biomass solids and the pretreated co-hydrolyzed biomass, i.e., without separation or washing of the solids. The hydrolyses were done using poplar wood, at pretreatment condition of 180° C. for 18 min and enzymatic conditions of 45 mg of cellulase protein/g of glucan in the original biomass and 15 mg of xylanase protein/g of xylan present in the raw biomass. The experiment was done in triplicate, the error bars represent the standard deviation.

FIG. 3 shows a CAD drawing of the metal well-plate in between a bottom and a top plate with a flat gasket for sealing purposes.

FIG. 4 is a photo showing the different parts needed to carry out the pretreatment and co-hydrolysis in a custom made 300 μL 96-well plate.

FIG. 5 illustrates top, side and bottom schematic views of a 96-well plate exemplary reactor.

FIGS. 6A-C illustrate top (A and B) and bottom (C) views of a 96-well plate exemplary reactor manufactured by electrical discharge machining or water jet machining.

FIG. 7 illustrates a 96-well plate embodiment of an exemplary reactor sized for use in a steam chamber.

FIG. 8 illustrates a clamp system for stacking multiple plates, as viewed at the top plate (A) or bottom plate (B) of the stack.

FIG. 9 illustrates multiple views of the 96-well plate exemplary reactor.

FIG. 10 illustrates multiple views of a partially assembled stack of 96-well reactors.

FIG. 11 illustrates multiple views of an assembled stack of 96-well reactors.

FIG. 12 is a graph showing total sugar release during pretreatment and co-hydrolysis carried out in a metal 96-well reactor. The same amount (standard deviation of 3.6%) of glucose and xylose is released from pretreated and co-hydrolyzed poplar wood across a complete row in a 96 well format.

### DETAILED DESCRIPTION

Cellulosic biomass may be pretreated in order to achieve high sugar and ethanol yields upon subsequent enzymatic treatment and fermentation. Among the most promising pretreatment technologies are dilute acid and water-only pretreatments. In both cases, an aqueous biomass slurry is heated to and kept at a certain temperature (e.g., 180° C.) for a certain time (e.g., 40 min) to open the biomass structure and release or break down certain compounds in the biomass (e.g., hemicellulose). During this procedure, substances inhibitory to enzymes and microorganisms are often introduced (e.g., added sulfuric acid), released from the biomass (e.g., acetic acid) or formed due to sugar and/or lignin decomposition (e.g., HMF). The presence of these inhibitory substances typically requires that the pretreated biomass slurries be separated and the solids be washed prior to further enzymatic hydrolysis to overcome negative effects that otherwise limit yields. Enzymes may then be added to the solid biomass fraction, which is re-suspended in a buffered solution (citric acid buffer, 0.05M final concentration), to hydrolyze the carbohydrate fraction and to release sugars that can subsequently be fermented to ethanol.

In current standard processes, the mixture and the amount of enzymes to be added are based on the composition of the washed solid fraction after pretreatment. Therefore, the pretreated solids have to be analyzed for their composition in an elaborate, manual wet-chemical approach (typically strong acid hydrolysis of the polymeric carbohydrate fraction). The ability to identify the optimum pretreatment conditions (optimum time for a certain pretreatment temperature) for different pretreatment methods (dilute acid or water-only) for many different feedstocks is thus difficult to achieve with current technologies. As a result, a new high-throughput (HTP) method for screening thousands of different biomass types for their advanced usability for ethanol production is needed.

One way to improve the efficiency of treating biomass samples is to pretreat a sample in a vessel prior to enzymatic treatment of the sample in the same vessel. This process, known as co-hydrolysis, is based on the observation that very dilute pretreatment slurries (e.g., 1 to 2% w/w) do not release or form a sufficiently high concentration of compounds that inhibit enzymes. Therefore, instead of separating the solids and the liquid after pretreatment, the slurry from pre-treatment is used in whole, thus avoiding filtration methods currently in use.

The concentration of the inhibitory substances is kept at a level low enough not to inhibit the subsequent enzymatic hydrolysis or at least to allow reasonable enzyme action to occur. The pretreated biomass slurry is neutralized if necessary, as in the case of dilute acid pretreatment, using a base (e.g., NaOH), and then a buffer and appropriate nutrients are added to the slurry to reach the same final concentration as for the separated and washed solids (e.g., 0.05M). This slurry is spiked with enzymes to break down the polymeric carbohydrate fractions in the pretreated biomass. The enzyme mixture for co-hydrolysis may be enriched with xylanase when a considerable amount of the xylan fraction may be left in the pretreated solids. Additionally, the concentration of soluble xylo-oligomers could reach enzyme inhibitory levels and may need therefore to be reduced. Xylanases and/or beta-xylosidases break down these oligomers in solution to monomeric xylose, which is not enzyme inhibiting.

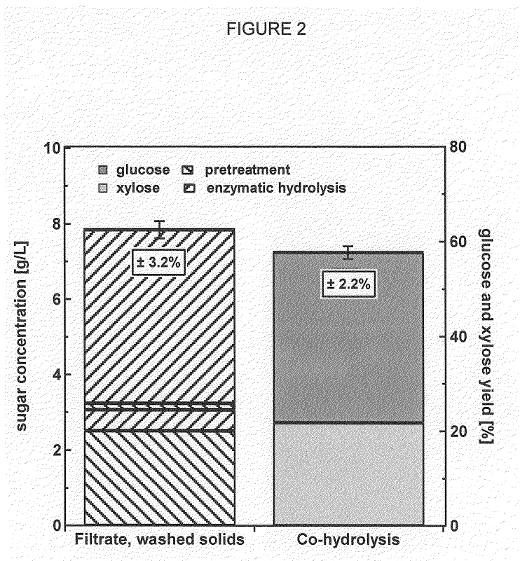

In the co-hydrolysis method, enzyme addition is based on composition of raw biomass. Since the compositional analysis of the biomass after pretreatment is not determined, the amount of enzymes to be added cannot be based on the glucan and xylan content in the pretreated solids. Thus, the amount of enzymes to be added for co-hydrolysis is based on the original glucan and xylan content in the raw biomass. This co-hydrolysis of pretreated dilute biomass slurries allows obtaining very similar results to the conventional filtering and washing procedure, particularly for higher enzyme loadings (see FIG. 2).

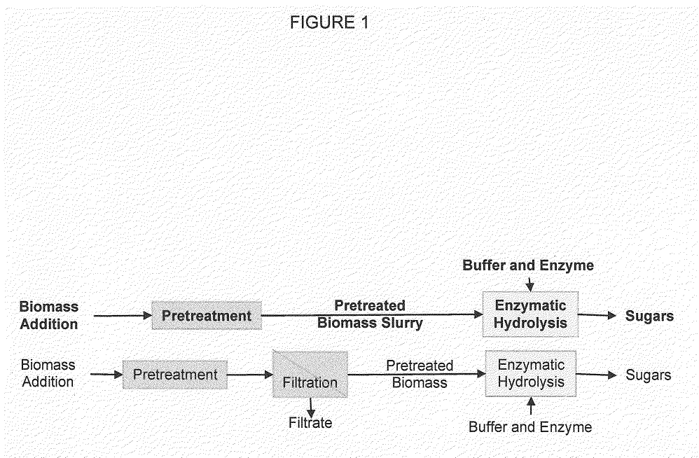

Although the co-hydrolysis approach greatly simplifies the procedure of pretreatment and enzymatic hydrolysis, this simplification is not enough to enable the screening of thousands of biomass types in a reasonable short time. Therefore, the disclosure provides a high-throughput (HTP) device that is based on co-hydrolysis in a 96-well plate format. Instead of separating the solid and the liquid fractions after pretreatment, the disclosure provides methods for using very dilute biomass slurries for the pretreatment step to reduce the problem of compounds inhibitory to enzymes and not to separate the solid and the liquid fractions as is customarily done (see FIG. 1).

Since standard plastic well-plates do not withstand the target temperatures during pretreatment (e.g., 180° C.), metal 96-well plate formats (described in greater detail below) were developed. The well-plate may feature the exact same outer dimensions and well diameter and well depth as a standard 300 μL well-plate from Corning. In one embodiment, the reactor comprises an aluminum bottom plate and free-standing corrosive resistant heat durable cup (e.g., Hastelloy cups). These well-plates may be sealed by using a sandwich configuration wherein the well-plate is clamped in between a thicker bottom and top plate, using a flat gasket (e.g., made of Viton or Silicone, 1/16″ thick) to seal each well individually. In another embodiment, the reactor is a single piece comprising a plate featuring 96 interconnected wells.

The reactors described below allow the pretreatment process to be carried out in very dilute (e.g., 1% to 2%) biomass slurries (whether in water or an acidic or basic catalyst solution), which reduces the concentration of compounds that may inhibit saccharification enzymes. Accordingly, enzymatic hydrolysis may be carried out in the same reactor without the need for steps such as liquid/solid separation or washing of solids prior to enzyme addition. This co-hydrolysis process simplifies the high-throughput analysis of multiple biomass samples. However, a proper comparison of multiple biomass samples requires that each sample be treated in a consistent manner. The exemplary reactors disclosed herein also allow for consistent pretreatment conditions for a large number of biomass samples.

In on embodiment, the reactor may be made of a thin (e.g., 2 mm thick aluminum) metallic heat conductive bottom plate onto which culture well (e.g., 96 wells) or cups are mounted. The wells are ideally non-corrosive and heat stable material or metal (e.g., Hastelloy cups to withstand 2% sulfuric acid). The metal well-plate and its contents may be heated using condensing steam or other means. The steam can freely flow around wells and condenses on the outer surface of the wells for heating purpose. In addition, condensing steam has a very high heat transfer coefficient, thereby heating up the plate very rapidly. The wells may be sealed (individually or together) by clamping the metal well-plate between a bottom (e.g., ⅜″ aluminum) and top plates with a flat gasket (e.g., made of Buna-N (Nitrile/NBR), Viton®(Fluorocarbon), Silicone, Chemraz®, EPDM/EPR, Kalrez®, Encapsulated (FEP or PFA), Teflon® (PTFE), Neoprene®, Fluorosilicone, Urethane, or AFLAS®) laid between the well-plate and the top plate. The well-plate can be clamped between the plates using any number of techniques. For example, the figures attached hereto demonstrate the use of four threaded studs in each corner of the bottom plate and four wing nuts, the well-plate is clamped together and tightly sealed.

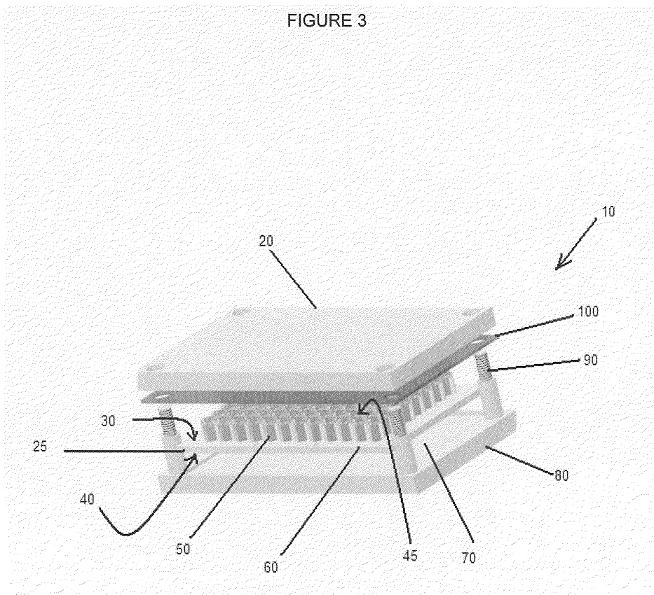

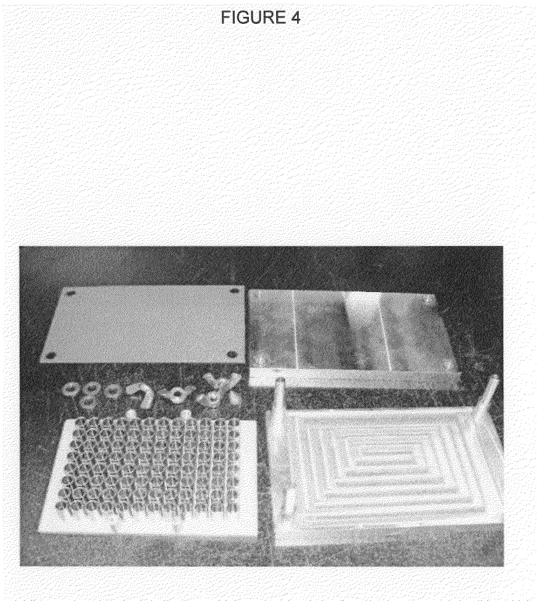

Referring now more particularly to the CAD drawing in FIG. 3, an assembly generally designated 10 comprises a one-piece metal lid 20, which is fabricated by conventional metal fabrication techniques employing the cutting, stamping and/or bending of sheet metal. Suitable metals include aluminum, steel, spring steel, stainless steel and stainless spring steel, preferably having a thickness between about 1 mm and 1.0 cm (e.g., 1.5-9.5 mm). The metallic design provides a high degree of chemical resistance and heat conductivity and durability. A planar, gasket 100 is depicted between the opening of the wells 50 and the bottom surface of the lid 20. The gasket is of sufficient area to fully engage the surface or a fraction of the surface of a multi-well plate. The gasket 100 is typically made from a material resistant to corrosion and degradation under high temperatures (e.g., Buna-N (Nitrile/NBR), Viton® (Fluorocarbon), Silicone, Chemraz®, EPDM/EPR, Kalrez®, Encapsulated (FEP or PFA), Teflon® (PTFE), Neoprene®, Fluorosilicone, Urethane, or AFLAS® or other thermoplastic polymer or elastomer. The gasket 100 can be manufactured using standard injection molding or extrusion technology, and may be affixed by an adhesive to the bottom surface of the lid 20. In one embodiment, the gasket is aligned by punching a hole or providing an identifying indication in at least one corner, which then fit over the studs and the spacers (see, e.g., FIG. 3). FIG. 3 shows well-plate 25 comprising a planar plate having a top 30 and bottom 40. Affixed to the top 30 are a plurality of wells 50 for retaining a liquid or slurry to be heated as described herein. The plate can be made of a metal that is heat conductive (e.g., aluminum or stainless steel) and is typically about 1-5 mm thick (e.g., about 2, 3, or 4 mm thick). The wells 50 comprise a bottom and at least one wall having an opening 45 for loading and removing material (e.g., a slurry). The wells are made of a corrosion resistant metal. The wells 50 and plate 25 may be manufactured as one unibody piece or the plate 25 and wells may be manufacture and subsequently attached to one another.

Also depicted in FIG. 3 is a bottom plate 80 comprising a clamping means for compressing the cover 20, gasket 100 and well-plate 25 and 50 together to seal openings 45. Such means are depicted in FIG. 3 as comprising threaded bolts, however other suitable means include clamps, vices and the like. The bottom plate 80 may be comprised of a metal (e.g., stainless steel, aluminum and the like). The bottom plate may be a porous material, an etched material or a ridged material that promotes flow of heat, air and water beneath or in contact with plate 25.

The biomass slurries in the well-plate may be heated by placing the reactor sandwich in a steam chamber where condensing steam can freely flow around the system and the 96 individual wells and rapidly and accurately heat the cups and their content. Alternatively, other devices such as a fluidized sand bath can be employed to heat the multiwell plate system.

An additional exemplary reactor comprises a 96-well reactor patterned on the Society for Biomolecular Screening (“SBS”) standard 96-well microtiter plate format in order to facilitate use in standard high-throughput robotics and instrumentation. Standard microtiter plates will not tolerate even mild pretreatment temperatures and standard sealing methods will not function under the pressures and temperatures of pretreatment. Moreover, reactors such as standard microtiter plates do not allow samples to be heated and cooled in a rapid manner wherein each sample well is heated or cooled in a consistent manner. The hardware and assay protocols described below provide a new way to handle these challenges and enable the high throughput pretreatment of thousands of biomass samples daily. While the discussion below focuses upon the 96-well plate exemplary reactor, additional multi-chamber formats known in the art, and the principles disclosed below are equally applicable to these multi-chamber formats.

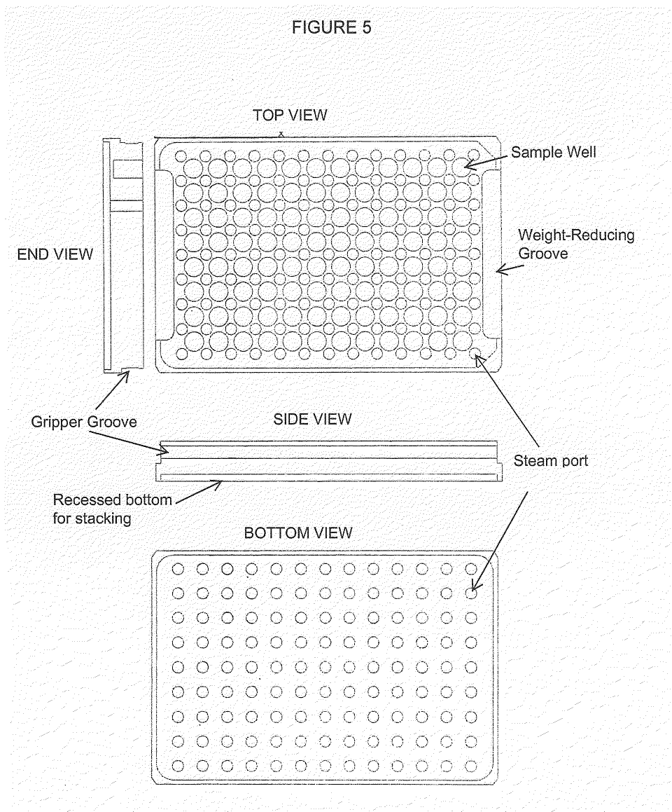

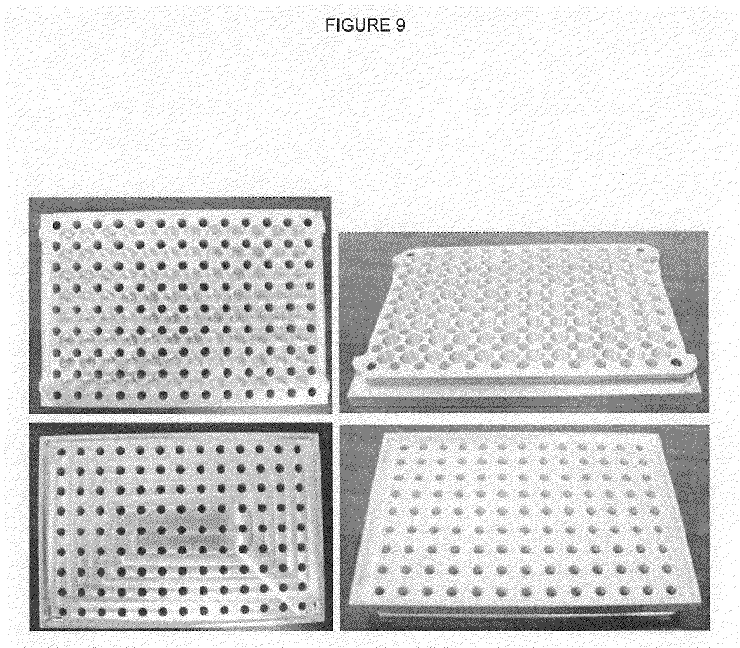

In general, the reactor comprises a plurality of sample wells of uniform size and distribution throughout the reactor (see, e.g., FIGS. 5 and 9). The sample wells are designed to maximize the transfer of heat applied to the reactor to the samples contained within the wells. The reactors further comprise a plurality of ports (e.g., steam channels) arrayed around the sample wells in order to maximize the outer surface area of the wells and thereby facilitate heat transfer. The ports may be introduced into the reactor by any standard machining means, such as mechanical machining, Electric Discharge Machining or high pressure water jet machining.

The reactor is constructed of material capable of withstanding the chosen pretreatment conditions of heat, chemistry, and pH. Typically, pretreatment temperatures range from about 100° C. to about 250° C., or from about 160° C. to about 220° C., while pretreatment pH values can be either acidic or basic, ranging from about 1 to about 13. In some embodiments, the reactor material can withstand temperatures of at least 120° C.

While any material that can withstand the pretreatment conditions may be employed, construction from metals is particularly suitable for typical biomass pretreatment conditions. As the reactor may be designed to be continuous from biomass dispensing through enzymatic digestion and analysis, the metallic construction may also be useful in dissipating static electricity when dispensing dry biomass. Suitable metals include aluminum, nickel, titanium, stainless steel, zirconium, and alloys of these metals. In some embodiments, the metals may include various coatings (such as electroplated gold, nickel, or in situ produced titanium oxide) that may augment the metallurgical resistance to pH. In certain embodiments, reactors may be constructed from a ceramic material such as silicon carbide. Metals or other materials that exhibit corrosion resistance, heat transfer efficiency, low density, and machinability are suitable as materials for reactors.

The reactor may also be constructed of carbon fiber, carbon nanotubes, or other carbon-based nanomaterial that exhibits rapid thermal conductivity and strength. These materials may be encased, molded, or extruded in a matrix of high temperature resin or other material designed to reinforce the carbon nanomaterial.

The reactor may also be coated with additional materials that, for example, increase the corrosion resistance of the reactor. Suitable coating materials include carbon nanotubes or other carbon-based nanomaterial, porcelain or other ceramic material, a diamond-like carbon coating, a high-temperature fluorocarbon coating, or Teflon™-impregnated nickel.

The reactor may be designed to minimize weight while retaining the structural rigidity required for efficient clamp sealing to prevent cross-contamination between adjacent wells during pretreatment. For example, a maximum reactor weight of 165 grams (which may be achieved, e.g., through the use of aluminum) will allow the use of a high-precision balance during biomass dispensing (e.g., by a Symyx powder-dispensing robot). Other balances or dispensing systems may require a different maximum weight, and the reactor may be designed to meet these specifications. Denser, more corrosion resistant metals, such as titanium or various stainless steel alloys (e.g., Hastelloy) may require the use of a balance capable of handling plates in excess of 500 grams. The metallic composition can also be labeled with numbers or barcodes by laser or acid etching in order to facilitate the identification of individual plates in a stack and to facilitate sample tracking by automation hardware. The reactor may also contain grooves or indentations that allow for handling by robotics (i.e., “gripper grooves”) or that reduce the weight of the reactor to allow for more accurate mass determinations.

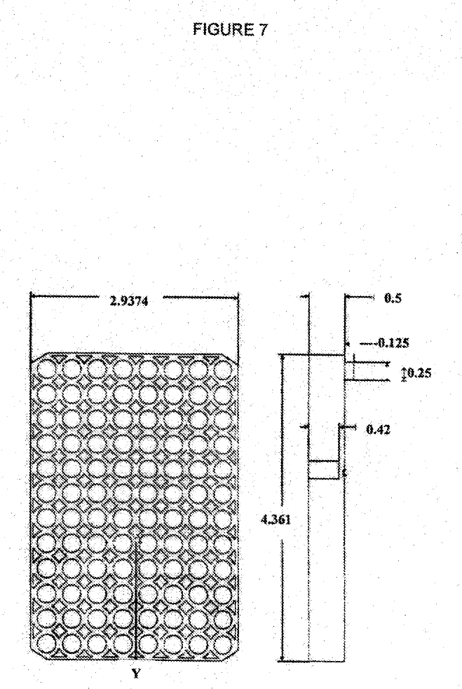

Maintaining the SBS standard plate footprint allows compatibility with standard plate and liquid handlers. However, in some embodiments, the SBS standard rectangular configuration of the block can be modified slightly to allow placement in various heating chambers. For a five-inch diameter steam chamber, for example, the plate may be modified by rounding off the corners in such a manner as to allow placement in the steam chamber but not to affect the spacing or dimensions of the wells (see FIG. 7). The block may also retain the original SBS 96-well microtiter plate footprint if a larger steam chamber such as a Parr reactor is utilized. Additional modifications may include slightly widening the diameter of the wells during machining in order to both allow for increased well volume capacity and further reduce the weight of the reactor block. Using a modified mill to round off the interior corner of the well such that the plating has a more uniform surface for adherence and to facilitate mixing and cleaning may be another useful alteration. Regardless of the heating chamber used, the block may also be designed with grooves and/or notches in the sides to allow various robotic grippers to handle the plate and excess metal removed to minimize weight and heat capacity (see FIGS. 5-7).

The reactor may be sized to accommodate larger or smaller volumes in each well. For example, the reactor may be deeper in order to increase well volume, or the well configuration may be altered to allow fewer but larger wells. Even larger volumes can be accommodated by a combination of fewer wells and deeper plates.

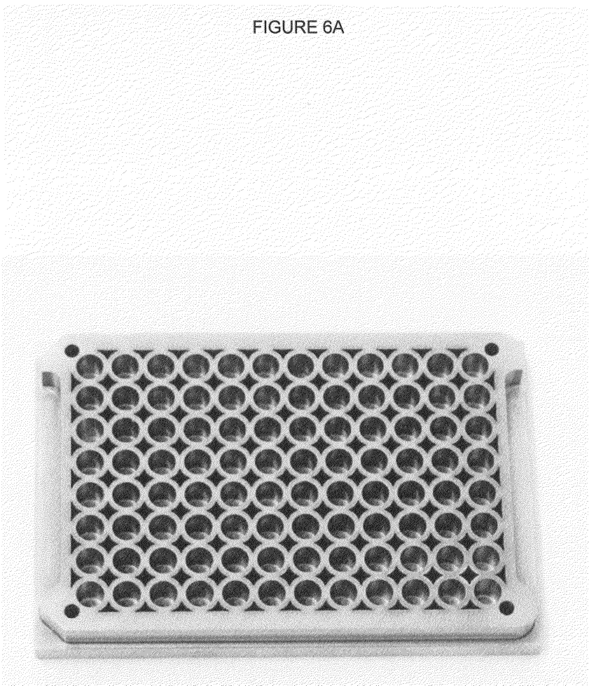

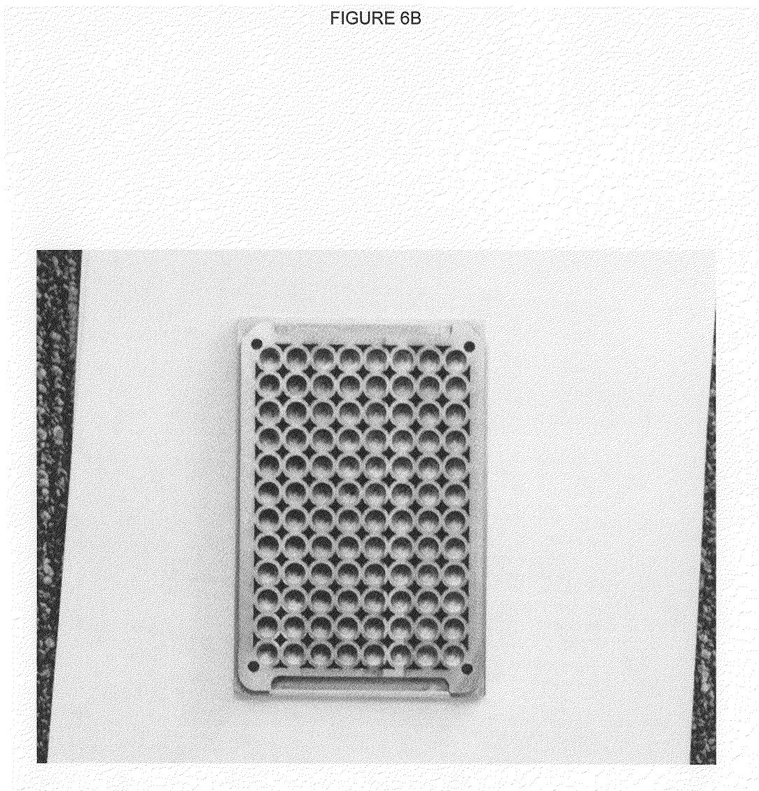

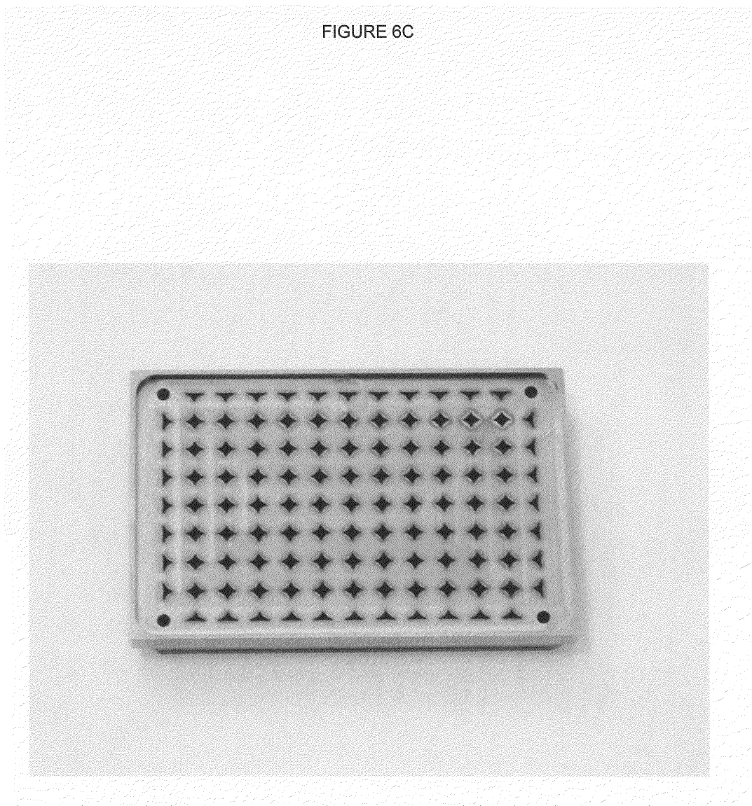

The reactor may be heated by any means known in the art, and may be designed for optimal compatibility with the chosen heating chambers. In some embodiments, the reactor may be heated and/or cooled in a steam chamber, such as, for example, a Parr reactor. In embodiments wherein the reactor is designed to operate in a steam chamber, the reactor may be constructed in such a manner that steam/air/water can circulate between the wells in order to facilitate rapid heat up and cool down of the reactor blocks. These steam channels may be cut into any size and shape that provides a consistent well wall thickness and the most uniform heat transfer and may be created by any machining technique known in the art. For example, the channels can be either circular for ease of machining using standard tools (see FIG. 5) or rounded diamond-cross-section using Electric Discharge Machining or high pressure water jet machining (FIGS. 6 and 7, respectively).

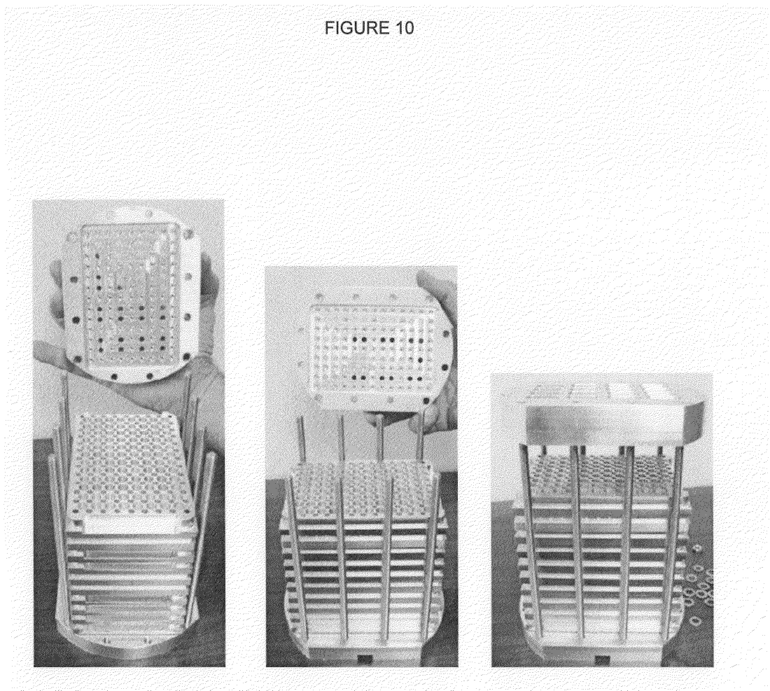

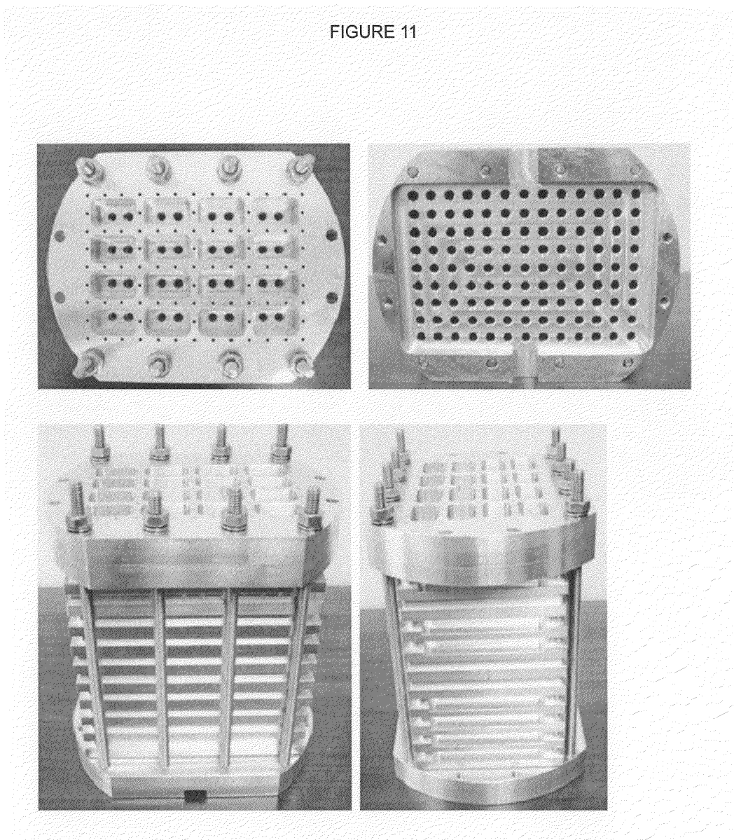

Although described above as a single reactor vessel, the exemplary reactor also encompasses multiple, interconnected reactors that allow for increased sample capacity. In certain embodiments, the reactor blocks may be designed to be stacked one upon one another with the ports or steam channels aligned to allow steam penetration through the entire stack. For either single reactors or stacks of multiple reactors, sample wells may be sealed with a gasket. Sample wells from each individual reactor may be sealed with a gasket perforated for the steam channels corresponding to those on each reactor vessel and compressed between adjacent reactors through an external clamping system (see FIGS. 10 and 11) designed to hold the entire stack of blocks under enough compression to seal the wells of each plate and prevent loss of well contents or dilution with steam.

The gasket may be made of any material that allows the wells of the reactor to be sealed yet withstands the heat, chemistry, and pH of the selected pretreatment conditions. The gasket should be sized (e.g., minimal thickness) so as to not impede the heat transfer of the sample wells. In certain embodiments, the gasket may be about 2.0 mm or less, 1.0 mm or less, or 0.5 mm or less. Suitable materials include synthetic polymers such as polytetrafluoroethylene (FIFE), Viton®, silicone, neoprene, rubber, Kal-Rez®, or similar inert materials. In some embodiments, the gasket may be 0.5 mm PTFE. Additionally, each plate may be individually sealed with a high-temperature aluminum foil-backed adhesive tape or seal that may be reinforced with glass fiber or cloth to facilitate removal. This may provide the advantage of minimal loss or liquid transfer from wells and the ability to centrifuge individual plates after pretreatment and cooling to minimize losses from condensation on the underside of the sealing film. The seal may be removed after pretreatment and centrifugation in order to add neutralization and enzyme mixes, or the seal may be pierced to add the reagents directly. The plate can then be resealed for enzyme digestion.

The gasket may also comprise an adhesive seal such as a metal (e.g., aluminum, copper, or similar) foil seal or a high-temperature sealing adhesive or film. Adhesive sealing films may also be used to enable centrifugation of plates to minimize liquid or condensate loss during disassembly. Adhesive sealing films can also be used to decrease volume loss during post-pretreatment incubations. The gasket materials described herein can also function in conjunction with a foil seal to increase sealing efficiency. The gasket material can also be reinforced with materials such as glass-impregnated PTFE.

The sealing gaskets or films can be precut with steam port holes or the holes may be cut after placement. The sealing gaskets may be separate from the reactor or attached to the top or bottom of each reactor.

Magnets may be inserted into some or all of the steam channels and used to sandwich the reactor plate between a magnetic (e.g., steel) top and bottom plate, thereby enhancing the seal and limiting water loss during enzyme incubation. The magnets can be free or affixed to a reactor. Suitable magnets include cylindrical neodymium magnets sized to fit within the steam ports of the reactor. The steel plates may also be coated with a thin fluorocarbon, silicon, rubber, or other coating to enhance sealing or eliminate the need for an adhesive seal.

Seals may be pre-pierced before enzyme addition in order to allow pipetting of enzyme and buffer into wells. Multiple piercing of each well can alleviate well overflow by allowing air to be displaced. The piercing system may employ a locating jig to align the piercing tool and plate. The jig may be adjustable to allow multiple piercings to be offset.

Since the entire assembly may be placed in a steam chamber for heating, the pressure difference between the sealed well and the external environment can be minimized. Upon post-pretreatment pressure release in the chamber, the entire stack can be rapidly cooled by submersion in a water bath. Interwell cooling provided by cooling liquid in the steam channels will minimize variation in pretreatment severity typically seen in non-ported heating blocks.

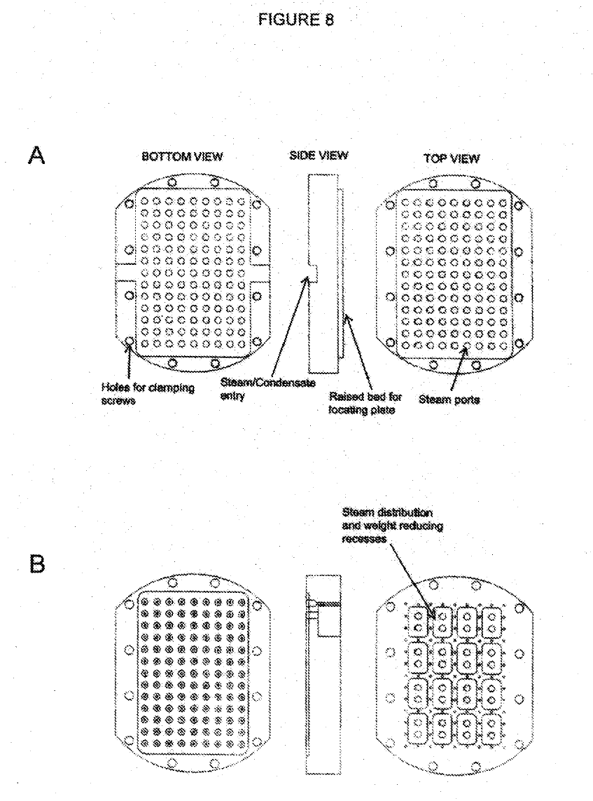

The stacked reactors may be held together with an external clamping system designed to hold the plates tightly to each other while allowing steam to penetrate the stack uniformly (FIGS. 8, 10 and 11). The end plates may also be machined to minimize weight (and therefore heat capacity) while maintaining the structural rigidity required to maintain even pressure across the plate stack. The central steam port may be sacrificed in order to provide a central compression point to keep the centers of the stacked plates in tight sealing proximity. In certain embodiments, the external clamping system may be a top and bottom plate with steam channels, along with clamping screws to fasten the stack together (see FIG. 11).

The clamping system may also comprise a center stud or studs threaded through one or more steam ports to tightly hold the center of the reactor plate stack. Alternatively, interlocking plate reactors comprising integral gaskets may be used, thereby allowing the stack to be assembled and sealed without the use of the external clamping system, reducing assembly/disassembly time. Such an arrangement may also reduce the overall heat capacity of the system, allowing faster heat up and cool down. Reactors with finely machined surfaces, such as those coated with a fluoropolymer, may provide for sealing without the need for a gasket material.

This devices and processes of the disclosure provide numerous advantages. For example, the reactors and methods described herein allow one to screen thousands of biomass types, pretreatment conditions and/or enzyme formulations in a much shorter time with much less manpower than by state-of-the-art procedures. The reactors also allow one to use very small amounts of biomass, thereby reducing the need to sacrifice plants for evaluations.

The pretreatment and enzymatic hydrolysis processes used for the production of fuel ethanol from cellulosic biomass can be greatly speeded up by using the sequential pretreatment and co-hydrolysis process of the disclosure accomplished in modified 96-well format reactors. The well format described herein is also advantageous due to the rapid heating and screening. The multiwell plate allows for heat to penetrate between the wells providing rapid and more uniform heating along with a better heat transfer coefficient.

The well-plate, clamped in between the bottom and top plate is heated to the target temperature by using condensing steam or other means. The reactor sandwich can then be placed in a heating device (e.g., an oven or in a steam reactor pressurized with condensing steam or in a fluidized sand bath) to increase the reactor and its contents to the target pretreatment temperature. An exemplary steam chamber may be assembled by using steam rated, readily available screw fitting, instruments and nipples.

The pretreatment reactions occurring in the reactor heated in the steam chamber can almost immediately be quenched by flash cooling the steam chamber and by subsequently rapidly injecting cooling water and thereby flooding the chamber to quickly decrease the temperature to ambient conditions.

The exemplary reactors described herein allow for rapid pretreatment of multiple biomass samples in order to evaluate the effect of the aforementioned factors on the pretreatability and subsequent enzyme digestibility of the biomass. One advantage of the exemplary reactors is the ability to carry out biomass allocation, pretreatment, conditioning, and enzyme digestion in a single reactor designed to meet the requirements of high temperature, corrosion resistance, rapid heat transfer, and sample containment required for a simple, process-driven, high throughput screening assay. This exemplary reactor also enables methods and systems for rapid, high-throughput pretreatment and subsequent enzyme hydrolysis testing of multiple biomass samples utilizing a novel pretreatment reactor system that is incorporated into the enzyme hydrolysis through unique hardware design and assay protocol steps.

The disclosure also includes methods for pretreating biomass. In general, these methods involve dispensing biomass into each sample well, adding water or an acidic or basic catalyst solution to each sample well, heating the reactor to temperatures of about 100° C. to 250° C., and terminating the pretreatment by cooling the reactor. An example of an acid treatment process is described in Aden et al. (National Renewable Energy Laboratory Report TP-510-32438 (2002)). In this process, dilute sulfuric acid (H₂SO₄) is added to biomass and the mixture is heated by direct steam injection to the desired temperature. The process may be carried out as a continuous or batch process. The reactors described herein may also be used for pretreatment methods followed by enzymatic saccharification protocols.

The disclosure includes systems and methods for the high-throughput pretreatment of biomass, and the high-throughput pretreatment methods may be utilized in conjunction with additional high-throughput processes and enzymatic assays. One example of an integrated system may be: 1) pre-processing biomass sample preparation (e.g., milling a biomass sample); 2) initial compositional analysis of each sample; 3) dispensing equal amounts of biomass samples to the wells of a reactor via a solids handling system (e.g., with a Symyx Powdernium robotics system); 4) dispensing water or catalyst solution to the wells via a liquids handling system (e.g., a Beckman-Coulter Biomek FX robotics system); 5) pretreating the biomass samples in a heating chamber (e.g., a steam chamber such as a Parr reactor); and 6) determining resulting sugar concentrations or conducting enzymatic digestions or assays on the pretreated biomass.

The above reactors may be utilized in conjunction with automated biochemical assays for determining the susceptibility of pretreated biomass to enzymatic digestion, as this is one of the more cost-intensive and rate limiting steps in the biomass-to-fuel process. The standard material preparation for enzymatic digestion screening involves liquid/solid separation, washing of solids, neutralization of catalyst (if needed), compositional analysis of the sample, and quantitative transfer of the samples to a suitable assay platform. All of these steps are exceptionally difficult to carry out on standard high-throughput platforms. The reactors and methods disclosed herein enable the high-throughput screening of biomass samples.

Specifically, the sealing of the wells during pretreatment will contain both the solids and liquids in each well. For acidic pretreatment regimens, a suitable base (e.g., NaOH) may be added to the sample wells to neutralize any acid catalyst (or acid to neutralize a basic catalyst for basic pretreatment regimens). A buffer may be added along with (or instead of) the base or acid in order to maintain an optimal pH level during enzymatic hydrolysis without any need for solid/liquid separation or washing. The reactor may be used as the enzyme digestion assay plate as well, eliminating the need for a quantitative transfer step. Enzyme loading may then be based on the composition of the original biomass, since all materials remain in the well, obviating the need for post-pretreatment chemical analysis.

The composition of these enzyme mixtures and the loading levels used can be adjusted and manipulated to evaluate several aspects of the sample attributes. High enzyme-to-substrate loadings (e.g., greater than 50 mg enzyme/g carbohydrate) can be used to evaluate potential extent of digestion. Low enzyme-to-substrate loadings (e.g., less than 5-15 mg cellulase/g carbohydrate) can be assessed to determine differences in rates of digestion between different samples. In addition to using cellulases to evaluate cellulose digestibility or xylanase to evaluate xylan hydrolysis, specific enzymes can be used either individually or in conjunction with defined activities to elucidate the most recalcitrant components or linkages in a given sample. Examples could include utilizing acetyl xylan esterase to determine the impact of post-pretreatment acetylation on digestibility, arabinofuranosidase or glucuronosidase activities to determine the synergy of xylan debranching with a commercial enzyme system, and ferulic acid esterase or lignin modifying enzymes to evaluate how lignin-xylan decoupling enhances cellulose and xylan digestion. As the number of enzyme activities involved in biomass hydrolysis numbers in the several dozens, the diagnostic capability of a high-throughput pretreatment/enzyme hydrolysis system increases dramatically as new activities are included. Sample aliquot tracking by the powder dispensing robot is useful to enable the downstream liquid handlers to dispense consistent enzyme-to-carbohydrate ratios in all wells regardless of the variation in biomass mass allocation. The same system that is used to seal and clamp multiple plates together during pretreatment can be used to enable efficient stacking of the plates during enzymatic digestion incubation.

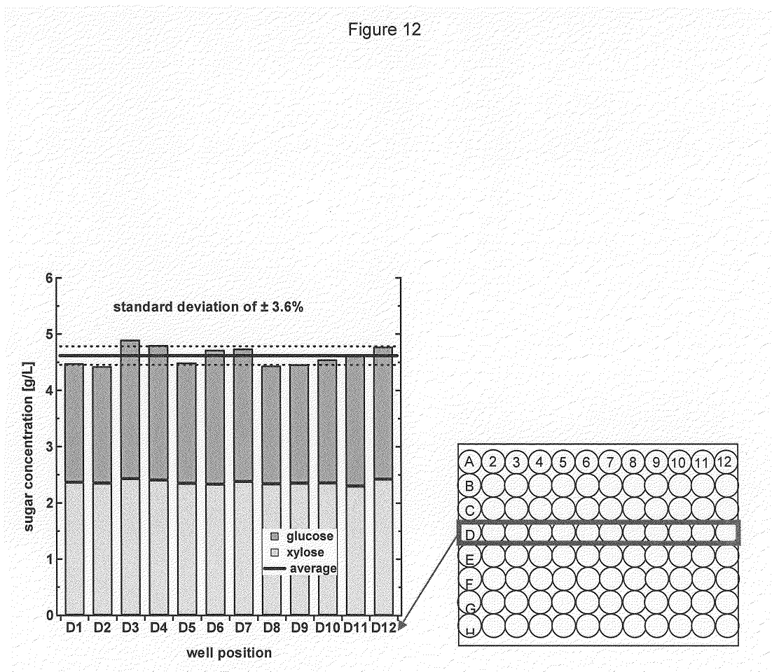

Initial experiments to evaluate pretreatment and co-hydrolysis using reactors disclosed herein have been carried out. The total xylose and glucose releases from poplar wood during pretreatment and enzymatic co-hydrolysis are shown for a complete row of 12 wells in a reactor (FIG. 12). The variation between individual wells is very small with the standard deviation being only 3.6%. The variation for the xylose release, which is mainly released during pretreatment, is smaller than for the glucose release, which is mainly released during enzymatic hydrolysis. This is an indication that the largest error is introduced by pipetting the enzyme mixture into the individual wells and not by weighing the biomass out or inhomogeneous pretreatment throughout the reactor.

The reactors described herein have additional uses apart from the co-hydrolysis of biomass. For example, the reactors may be used for high-temperature materials testing, for combinatorial chemistry high temperature reactions, or for temperature stability studies on chemicals, pharmaceuticals, agricultural chemicals, food products, etc. The reactors may be also be used in decompositional studies of materials (such as, e.g., 2-stage acid hydrolysis of biomass for compositional analysis) or other non-high temperature or non-corrosive applications where rapid temperature changes are desired. Additional uses include high throughput PCR applications or other application where rapid and frequent temperature changes are required of multiple small reaction volumes.

As used herein, “biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover or fiber, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, corn fiber, grasses, wheat, wheat straw, hay, rice straw, switchgrass, waste paper, sugar cane bagasse, sorghum stalks, soy hulls or stalks, components obtained from milling of grains, trees, branches, roots, leaves, wood chips, sawdust, shrubs and bushes, vegetables, fruits, flowers and ruminant animal manure. In one embodiment, biomass that is useful for the exemplary reactor includes biomass that has a relatively high carbohydrate value, is relatively dense, and/or is relatively easy to collect, transport, store and/or handle. In another embodiment of the exemplary reactor, biomass that is useful includes corn cobs, corn stover, corn fiber, and sugar cane bagasse.

The exemplary reactors, in various embodiments, includes components, methods, processes, systems and/or apparatus substantially as depicted and described herein, including various embodiments, sub combinations, and subsets thereof. Those of skill in the art will understand how to make and use the present exemplary reactors after understanding the present disclosure. The exemplary reactors, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the exemplary reactors and methods related thereto has been presented for purposes of illustration and description. The foregoing is not intended to limit the exemplary reactors to the form or forms disclosed herein. In the foregoing Detailed Description for example, various features of the exemplary reactors are grouped together in one or more embodiments for the purpose of streamlining the disclosure. This method of disclosure is not to be interpreted as reflecting an intention that the claimed exemplary reactors require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the exemplary reactors.

Moreover though the description of the exemplary reactors has included descriptions of one or more embodiments and certain variations and modifications, other variations and modifications are within the scope of the exemplary reactors, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A multi-chamber reactor for high throughput screening of biomass samples, comprising:
a plurality of wells for holding biomass samples; and
a plurality of ports located around the sample wells, wherein the ports allow for rapid and even heating and cooling of the sample wells.

2. The multi-chamber reactor of claim 1, wherein the plurality of wells for holding biomass samples comprises a plate comprising 96 wells and a plurality of ports disposed around the 96 wells.

3. The multi-chamber reactor of claim 1, wherein the reactor further comprises a top plate and a bottom plate, and wherein the plurality of wells for holding biomass samples are composed of a non-corrosive heat stable material and are disposed between the top plate and bottom plate.

4. The multi-chamber reactor of claim 1, wherein the reactor further comprises a top plate and a bottom plate, and wherein the plurality of wells for holding biomass samples comprise cups composed of a non-corrosive heat stable material affixed to the bottom plate.

5. The multi-chamber reactor of claim 4, further comprising a means for compressing the top plate to the plurality of wells to form a seal.

6. The multi-chamber reactor of claim 1, wherein the reactor is constructed of a material that can withstand temperatures of at least 120° C. and pH values of about 1 to about 13.

7. The multi-chamber reactor of claim 1, wherein the reactor is constructed of a metal, ceramic material, or carbon-based nanomaterial.

8. The multi-chamber reactor of claim 7, wherein the reactor is constructed of aluminum, nickel, titanium, stainless steel, or any alloy thereof.

9. The multi-chamber reactor of claim 7, wherein the reactor further comprises a coating of gold, nickel or titanium oxide.

10. The multi-chamber reactor of claim 1, further comprising an external clamping system.

11. The multi-chamber reactor of claim 10, wherein the reactor further comprises at least one gasket.

12. A method for high throughput screening of biomass samples, comprising:
a) placing at least one biomass sample into one or more wells of at least one multi-chamber reactor;
b) adding water or catalyst solution to the wells of the reactor; and
c) heating the reactor.

13. The method of claim 11, further comprising adding at least one enzyme to the reactor wells to hydrolyze the biomass sample.

14. A method for treating biomass samples, comprising:
a) providing a slurry of cellulosic biomass to one or more wells of at least one multi-chamber reactor; and
b) incubating the reactor at a temperature sufficient to open the biomass structure and release or break down hemicelluloses.

15. The method of claim 14, wherein the slurry is a dilute slurry of about 1 to 2% w/w.

16. The method of claim 14, further comprising adjusting the pH of the slurry prior to incubation.

17. The method of claim 14, wherein reactor is incubated at a temperature between about 120 and 200 degrees C.

18. The method of claim 14, further comprising removing the biomass after incubation and extracting the sugar.

19. The method of claim 14, wherein incubating the reactor comprises contacting the reactor with hot air, steam, hot sand, convection heat, or hot oil.

20. A system for high throughput screening of biomass samples, comprising:
at least one multi-chamber reactor according to claim 1; and
a steam chamber.
